(19) United States
(12) Patent Application Publication
(10) Pub. No.: US 2023/0099027 A1
(43) Pub. Date: Mar. 30, 2023

(54) VIRUCIDAL COMPOSITIONS AND USE THEREOF

(71) Applicant: Ecole Polytechnique Fédérale de Lausanne (EPFL), Lausanne (CH)

(72) Inventors: Francesco STELLACCI, Morges (CH); Ozgun KOCABIYIK, Cambridge, MA (US); Caroline TAPPAREL VU, Chêne-Bougeries (CH); Valeria CAGNO, Vinzel (CH); Paulo Jacob SILVA, Denges (CH)

(21) Appl. No.: 17/936,550

(22) Filed: Sep. 29, 2022

Related U.S. Application Data
(63) Continuation of application No. PCT/EP2021/058086, filed on Mar. 29, 2021.

(30) Foreign Application Priority Data
Mar. 30, 2020 (EP) 2016-6899.3
Mar. 30, 2020 (EP) 20166899.3

(51) Int. Cl. A61K 31/724; A61P 31/14

(52) U.S. Cl. CPC A61K 31/724; A61P 31/14

(57) ABSTRACT

The invention relates to virucidal compositions comprising a sialic acid (SA) moiety, pharmaceutical compositions and disinfection and/or sterilization compositions comprising thereof and uses thereof in disinfection and/or sterilization methods and in treating and/or preventing COVID-19 and other respiratory diseases caused by coronaviruses and/or influenza viruses.

### FIELD OF THE INVENTION

The invention relates to virucidal compositions comprising a sialic acid (SA) moiety and the uses thereof including against COVID-19 caused by SARS-CoV-2 and against Influenza. The invention further relates to pharmaceutical compositions and disinfection and/or sterilization compositions comprising a virucidal composition and uses thereof in disinfection and/or sterilization methods and in treating and/or preventing COVID-19 and other respiratory diseases caused by coronaviruses and/or influenza virus

### BACKGROUND OF THE INVENTION

Viruses are the most abundant biological entities on Earth and are capable of infecting all types of cellular life including animals, plants, bacteria and fungi. Viral infections kill millions of people every year and contribute substantially to health care costs. The negative impact viruses can have on society is significant. From viral infections of food, crops and livestock, to the serious health impacts viral infections, such as SARS-CoV-2, HIV, Ebola, Zika or Influenza viruses, have on humans. COVID-19 hit the world stage at the end of 2019 and reached pandemic designation in early March 2020. Indeed, severe acute respiratory syndrome coronavirus 2 (SARS-CoV-2), the causative agent of coronavirus disease 2019 (COVID-19) was first reported in December 2019. Since then, SARS-CoV-2 has emerged as a global pandemic with an ever-increasing number of severe cases requiring specific and intensive treatments that threatens to overwhelm healthcare systems.

The best way to fight viral infection is vaccination. However, vaccines are not always available and in underdeveloped countries having sufficient vaccine coverage can be a significant challenge. Additionally, once infected, vaccination is no longer useful and drugs are needed to help the immune system to fight the infection. Anti-viral drugs, which act by disrupting the intracellular pathways used by viruses to replicate, are often prescribed to aid the immune system's fight against the infection. Indeed, there are now several vaccines in clinical trials that demonstrate a high level of efficacy, however there is still no data indicating the durability of this vaccine induced protection. Thus in the ongoing COVID-19 pandemic, there is a large unmet medical need for therapeutic interventions that can protect at-risk individuals, be of significant importance to protect individuals that are less able to mount an effective anti-SARS-CoV-2 immune response following vaccination and to treat those already infected with the virus.

Influenza viruses are among the most infective viruses. Every year different influenza strains infect a large fraction of both the animal and human population, endangering infants, the elderly and immunocompromised people, all having a risk of hospitalization and death due to influenza-related complications. As a result, seasonal influenza poses remarkable impacts on socio-economy. In fact, respiratory diseases can cost a significant fraction of the total health expenditures in developed and mainly in developing countries. Because influenza mutates so rapidly, the development of a vaccine is still a major challenge. Vaccine development would pose even higher challenges when focused on the occasional pandemics instead of yearly outbreaks. In such case, the development time of a new vaccine, which is on average 6 months, would represent a serious risk. Furthermore, even in the presence of a vaccine, reaching a reasonable vaccination coverage is far from a foregone conclusion. Therefore, the risk of a new pandemic, such as the Spanish Flu, is still present and recognised as one of the top threats to global health.

There remains an unmet need for antiviral drugs against viruses, such as SARS-CoV-2 and influenza. An ideal anti-viral drug should be broad-spectrum, target a highly conserved part of the virus, have an irreversible effect, i.e. be virucidal (in order to avoid loss of efficacy due to the dilution in body fluids) at low concentrations, and obviously be non-toxic.

### SUMMARY OF THE INVENTION

The present invention provides cyclodextrin-based compositions that are effective in treating diseases caused by coronaviruses and/or influenza viruses.

An aspect of the present invention provides a virucidal composition comprising a core and a plurality of ligands covalently linked to the core, wherein at least a portion of said ligands comprise a sialic acid moiety and wherein:
the core is cyclodextrin, and
the ligands are the same or different and are optionally substituted alkyl-based ligands.

The ligands are bound via the —OH moieties on the primary face of the cyclodextrin; which can remain —OH or be —SH where unsubstituted by a ligand.

Another aspect of the present invention provides a virucidal composition represented by Formula (I)

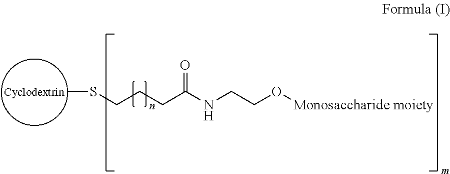

wherein
m is 2 to 8,
n is 2 to 28 or 4 to 13,
and sialic acid (SA) is the monosaccharide moiety.

It will be understood by those skilled in the art that the oxygen atom shown adjacent the monosaccharide moiety in Formula (I) can be considered part of the sialic acid.

Another aspect of the present invention provides a virucidal composition represented by Formula (II)

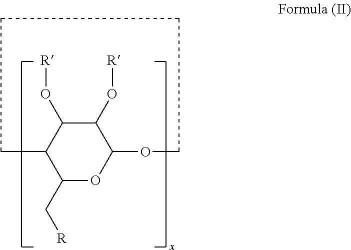

wherein:
each R, independently, is OH, SH or an optionally substituted alkyl-based ligand, wherein no more than 4 R groups can be OH or SH and at least two of said ligands have a sialic acid moiety;
each R′, independently, is H, —(CH₂)_{y}—COOH, —(CH₂)_{y}—SO⁻₃, a polymer or a water solubilizing moiety;
x is 6, 7 or 8; and
y is an integer from 4 to 20,

or a pharmaceutically acceptable salt thereof.

Still another aspect of the invention entails the compounds SA11 and SA6, both of which correspond to Formula (III) as shown below:

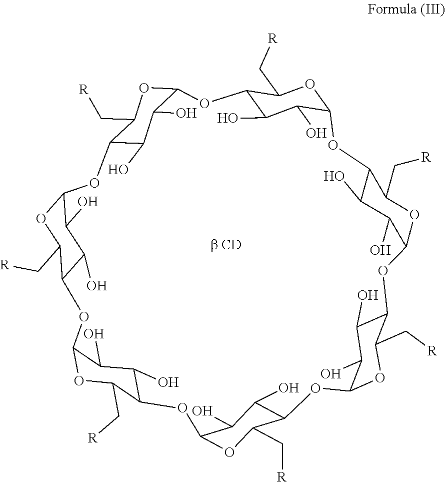

where R is selected from —OH, —SH, Formula (IV), Formula (V), Formula (VI) and Formula (VII):

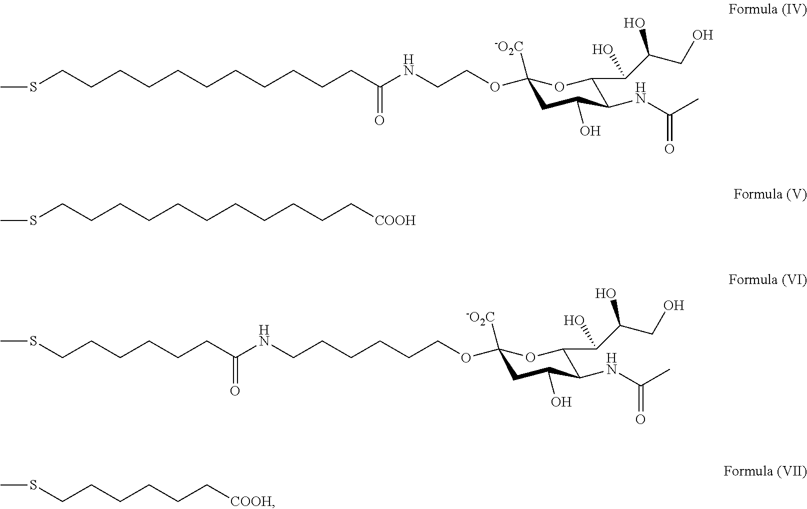

or a pharmaceutically acceptable salt thereof.

For SA11:
from 2 to 7 (especially 3 to 7, 4 to 7, or 4 to 6) R groups are represented by Formula (IV), and
from 5 to 0 (especially 4 to 0, 3 to 0, or 3 to 1) R groups are represented by Formula (V),
with 0, 1 or 2 R groups being —OH or —SH.

For SA6:
from 2 to 7 (especially 3 to 7, 4 to 7, or 4 to 6) R groups are represented by Formula (VI), and
from 5 to 0 (especially 4 to 0, 3 to 0, or 3 to 1) R groups are represented by Formula (VII),
with 0, 1 or 2 R groups being —OH or —SH.

A further aspect of the present invention provides a pharmaceutical composition comprising an effective amount of one or more virucidal compositions of the present invention and at least one pharmaceutically acceptable excipient, carrier and/or diluent.

A further aspect of the present invention provides the virucidal composition of the present invention for use in treating and/or preventing COVID-19, influenza virus infections and/or diseases associated with influenza viruses.

A further aspect of the present invention provides a virucidal composition comprising an effective amount of one or more virucidal compositions of the present invention and optionally at least one suitable aerosol carrier.

A further aspect of the present invention provides a method of disinfection and/or sterilization comprising using the virucidal compositions of the present invention, or a virucidal composition of the present invention.

A further aspect of the present invention provides a device comprising one or more virucidal compositions of the present invention and means for applying or dispensing the virucidal composition or compositions.

A further aspect of the present invention provides a use of the virucidal compositions of the present invention or the virucidal composition of the present inventions for sterilization and/or for disinfection.

### BRIEF DESCRIPTION OF THE FIGURES

FIG. 1: Dose-array response for the inhibition of a VSV-Sars-CoV2 expressing the Sars-CoV-2 Spike protein (a pseudo-virus containing the Spike S proteins of SARS-CoV-2).

FIG. 2 illustrates the results of testing compositions of the invention for inhibition of the Netherland 09 strain of H1N1 (influenza A).

### DETAILED DESCRIPTION OF THE INVENTION

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

### Definitions

In the case of conflict, the present specification, including definitions, will control.

Unless defined otherwise, all technical and scientific terms used herein have the same meaning as is commonly understood by one of skill in art to which the subject matter herein belongs. As used herein, the following definitions are supplied in order to facilitate the understanding of the present invention.

As used in the specification and claims, the singular forms “a”, “an” and “the” include plural references unless the context clearly dictates otherwise.

As used herein, the term “alkyl” refers to a straight hydrocarbon chain containing from 1 to 50 carbon atoms, preferably 4 to 30 carbon atoms. Representative examples of alkyl include, but are not limited to methyl, ethyl, propyl, butyl, pentyl, hexyl, heptyl, octyl, nonyl, decyl, . . . .

As used in the specification and claims, the term “and/or” used in a phrase such as “A and/or B” herein is intended to include “A and B”, “A or B”, “A”, and “B”.

As used herein, the term “carboxyalkyl” refers to a carboxy group appended to the parent molecular moiety through an alkyl group as defined herein.

As used in the specification and claims, the term “at least one” used in a phrase such as “at least one C atom” can mean “one C atom” or “two C atoms” or more C atoms.

As used herein, the term “biocompatible” refers to compatibility with living cells, tissues, organs, or systems, and having no significant risk of injury, toxicity, or rejection by the immune system.

The term “comprise” is generally used in the sense of include, that is to say permitting the presence of one or more features or components. In addition, as used in the specification and claims, the language “comprising” can include analogous embodiments described in terms of “consisting of” and/or “consisting essentially of”.

As used herein, “influenza” refers to sialic acid-seeking, airborne transmissible (human or animal) RNA viruses, such as influenza A virus, influenza B virus, influenza C virus and influenza D virus. Influenza A virus encompasses the following serotypes: H1N1, H2N2, H3N2, H5N1, H7N7, H1N2, H9N2, H7N2, H7N3, H10N7, H7N9, and H6N1.

“Mammal” for purposes of treatment refers to any animal classified as a mammal, including humans, domestic and farm animals or pet animals, such as dogs, horses, cats, cows, monkeys etc. Preferably, the mammal is human.

As used herein, “nano”, such as used in “nanoparticle”, refers to nanometric size, such as a particle having a nanometric size, and is not intended to convey any specific shape limitation. In particular, “nanoparticle” encompasses nanospheres, nanotubes, nanoboxes, nanoclusters, nanorods and the like. In certain embodiments the nanoparticles and/or nanoparticle cores contemplated herein have a generally polyhedral or spherical geometry.

As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. Other animals, such as a chicken, are also encompassed by these terms. In preferred embodiments, the terms “subject” or “patient” refer to a human and animals, such as dog, cat, rat, mouse, monkey, cow, horse, goat, sheep, pig, camel, chicken. In some embodiments, the subject is a subject in need of treatment or a subject being infected by SARS-CoV-2 or other coronaviruses. In other embodiment, a subject can be an animal infected by avian influenza, such as a chicken. However, in other embodiments, the subject can be a healthy subject or a subject who has already undergone treatment. The term does not denote a particular age or sex. Thus, adult, children and newborn subjects, whether male or female, are intended to be covered.

The term “therapeutically effective amount” refers to an amount of a virucidal composition of the invention effective to alter SARS-CoV-2, another coronavirus or influenza virus, and to render it inert, in a recipient subject, and/or if its presence results in a detectable change in the physiology of a recipient subject, for example ameliorates at least one symptom associated with a viral infection, prevents or reduces the rate transmission of at least one viral agent.

“Treatment” or “treating” refers to both therapeutic treatment and prophylactic or preventative measures. Those in need of treatment include those already being infected by SARS-CoV-2, another coronavirus or influenza virus, as well as those in which the viral infection is to be prevented. Hence, the mammal, preferably human, to be treated herein may have been diagnosed as being infected by a virus, or may be predisposed or susceptible to be infected by a virus. Treatment includes ameliorating at least one symptom of, curing and/or preventing the development of a disease or condition due to viral infection and/or preventing the number of people contaminated by an infected subject. Preventing is meant attenuating or reducing the ability of a virus to cause infection or disease, for example by affecting a post-entry viral event.

As used herein, the term “virucidal” refers to a characterization of antiviral efficacy determined by in vitro testing demonstrating irreversible inhibition of the infectivity of a virus following interaction with an antiviral compound or composition. The interaction inhibits infectivity, for example, by binding to the virus or otherwise interfering with the virus' surface ligands. However, even following termination of the interaction (for example, by dilution) and absent any added materials or conditions promoting viral reconstitution, it is essentially impossible for the virus to resume infectivity. Interaction with antiviral compound or composition alters the virus, rendering it inert, and thereby prevents further infections.

As used herein, the term “virustatic” refers to a characterization of antiviral efficacy determined by in vitro testing demonstrating reversible inhibition of the infectivity of a virus following interaction with an antiviral composition. The interaction inhibits infectivity, for example, by binding to the virus or otherwise interfering with the virus' surface ligands. However, once the interaction terminates (for example, by dilution) and absent any added materials or conditions promoting viral reconstitution, it is possible for the virus to resume infectivity.

The term “water solubilizing moiety” refers to a group appended to the parent molecular moiety, which increases the aqueous solubility of the overall composition; if replaced by a hydrogen the overall composition will be less soluble at micromolar concentrations. Water soluble moieties include ketones, alcohols, aldehydes, ethylene glycols and charged groups such as amines, carboxylates, phosphates, sulfates and sulfonates.

Compositions

In testing the effectiveness of earlier compositions (as described in pending applications WO2018/015465 and WO2020/048976, incorporated herein by reference) against COVID-19 a newly synthesized composition was included in the tests and surprisingly showed efficacy. Further, it has surprisingly been discovered that influenza does not develop resistance against such newly synthesized composition.

An embodiment of the present invention provides a virucidal composition comprising a core and a plurality of ligands covalently linked to the core, wherein at least a portion of said ligands comprise a sialic acid moiety and wherein:
the core is cyclodextrin, preferably selected from the group comprising alpha-cyclodextrin, beta-cyclodextrin, gamma-cyclodextrin or combinations thereof, and
the ligands are the same or different and are optionally substituted alkyl-based ligands, linked to the primary face of the cyclodextrin, preferably optionally substituted C₄-C₃₀ alkyl-based ligands, more preferably optionally substituted C₆-C₁₅ alkyl-based ligands.

The ligands are bound via the —OH moieties on the primary face of the cyclodextrin; which can remain —OH or be —SH where unsubstituted by a ligand.

The virucidal compositions of the present invention can be purified single molecules or compounds which are also intended to be encompassed within the scope of the present invention.

Cyclodextrins (CDs) are naturally occurring cyclic glucose derivatives consisting of α(14)-linked glucopyranoside units. Their cyclic structure creates a truncated cone shape with the primary hydroxyls of the glucose units on the narrow face and the secondary hydroxyls on the wider face. Each face can be readily and independently functionalised. The most commonly used natural CDs have 6, 7, and 8 glucopyranoside units, referred to as alpha (“α”), beta (“β”) and gamma (“γ”) cyclodextrin, respectively. The preferred cyclodextrin is beta. Because of the cyclic structure of CDs, they have a cavity capable of forming supramolecular inclusion complexes with guest molecules. As CDs are naturally occurring, readily functionalised, have a cavity for guest inclusion and are biocompatible, they have found use in many commercial applications including drug delivery, air fresheners, etc. The difference in reactivity of each face of CDs has been used for the synthesis of a wide range of modified cyclodextrins. The primary face of CDs is more readily modified, with control over the degree and location of substitution being possible. CD derivatives that bear a good leaving group, such as halogenated CDs, are important intermediates in CD functionalisation. By replacing all of the primary hydroxyl units of CDs with iodo-units gives an intermediate that allows for complete functionalisation of the primary face, whilst leaving the secondary hydroxyls and the rigid truncated cone shape intact. In one embodiment, heptakis-6-iodo-6-deoxy-beta-cyclodextrin was synthetized followed by reaction with mercaptoundecaosulphonate (MUS) to yield a CD functionalised on the primary face with undecanaosulfonate groups. It is then possible to independently modify the secondary face of the cyclodextrin to introduce further solubilising groups, dye molecules, polymers, etc. Moreover, the size of β-CD (diameter of approximately 1.5 nm) falls within the preferred nano size for cores of the invention and matches well with the HA globular head (˜5 nm). Beta-cyclodextrin has a rigid chemical structure that is believed to contribute to virucidal activity, and can have maximum of 7 sialic acid-bearing ligands depending from the narrow face, preferably 3 to 4 sialic acid-bearing ligands.

The ligands (or ligand compounds) of virucidal compositions of the invention are typically sufficiently long optionally substituted alkyl-based ligands (C₄-C₃₀ or preferably C₆-C₁₅) to present SA for binding with a virus and are hydrophobic.

Typically, in the context of the present invention, the optionally substituted alkyl-based ligands are selected from the group comprising hexane-, pentane-, octane-, undecane-, hexadecane-based ligands.

Optionally substituted alkyl-based ligands, substituted C₄-C₃₀ alkyl based ligands, and substituted C₄-C₃₀ carboxyalkyls of virucidal compositions of the present invention can be selected from and/or optionally substituted with 1, 2, 3, 4 or 5 substituents independently selected from the group comprising: alkenyl, alkenylthio, alkenyloxy, alkoxy, alkoxyalkoxy, alkoxyalkoxyalkoxy, alkoxyalkoxyalkyl, alkoxyalkoxyalkylthio, alkoxyalkyl, alkoxycarbonyl, alkoxycarbonylalkoxy, alkoxycarbonylalkyl, alkoxysulfonyl, alkyl, alkylamidoalkyl, alkylamidoalkoxy, alkylamidoalkylthio, alkylamidoalkyl-polyethoxy-alkylthio, alkylamidoalkyl-polyethoxy-alkyoxy, alkylamidoalkyl-polyethoxyalkyl-pyrrolidine-2,5-dione-thio, alkylamido-alkyl-polyethoxyalkyl-pyrrolidine-2,5-dione-oxy, alkylcarbonyl, alkylcarbonylalkoxy, alkylcarbonylalkyl, alkylcarbonylalkylthio, alkylcarbonyloxy, alkylcarbonylthio, alkylsulfinyl, alkylsulfinylalkyl, alkyl sulfonyl, alkylsulfonylalkyl, alkylthio, alkylthio alkyl, alkylthioalkoxy, alkynyl, alkynyloxy, alkynylthio, aryl, arylcarbonyl, aryloxy, arylsulfonyl, carboxy, carboxyalkoxy, carboxyalkyl, carboxyalkylthio, cyano, cyanoalkoxy, cyanoalkyl, cyanoalkylthio, 1,3-dioxolanyl, dioxanyl, dithianyl, ethylenedioxy, formyl, formylalkoxy, formylalkyl, haloalkenyl, halo alkenyloxy, haloalkoxy, haloalkyl, haloalkynyl, halo alkynyloxy, halogen, heterocycle, heterocyclocarbonyl, heterocyclooxy, heterocyclosulfonyl, hydroxy, hydroxyalkoxy, hydroxyalkyl, mercapto, mercapto alkoxy, mercapto alkyl, methylenedioxy, nitro, polyethylene glycol, polyethylene glycol pyrrolidine-2,5-dione-thio, and polyethylene glycol pyrrolidine-2,5-dione-oxy. Preferably, the substituted alkyl-based ligands are selected from the group: alkylamidoalkoxy, alkylamidoalkylthio, carboxyalkyloxy, and carboxyalkylthio.

Preferably, substituted alkyl-based ligands, substituted C₄-C₃₀ alkyl-based ligands, and substituted C₄-C₃₀ carboxyalkyls are substituted with one mercapto group. Preferred substituted alkyl-based ligands are C₄-C₃₀, or C₆-C₂₀, or C₆-C₁₅, or C₈-C₁₃ alkylamidoalkoxy or alkylamidoalkylthio; more preferably —S—(CH₂)ₐ—C(O)—NH—(CH₂)_{b}— or —O—(CH₂)ₐ—C(O)—NH—(CH₂)_{b}— where a is 4 to 15, b is 1 to 10 and a+b is 6 to 20, and still more preferably where a is 6 to 13, b is 2 to 8 and a+b is 9 to 15.

In some embodiments of the invention, the plurality of ligands of the invention comprises a mixture of at least two structurally different ligands, such as a polyethylene glycol, polyethylene glycol pyrrolidine-2,5-dione-thio, polyethylene glycol pyrrolidine-2,5-dione-oxy, carboxyalkyloxy, carboxyalkylthio, alkylamidoalkoxy, alkylamidoalkylthio, alkylamidoalkyl-polyethoxy-alkylthio, alkylamidoalkyl-polyethoxy-alkyoxy, alkylamidoalkyl-polyethoxyalkyl-pyrrolidine-2,5-dione-thio, or alkylamidoalkyl-polyethoxyalkyl-pyrrolidine-2,5-dione-oxy. The term “mixture of at least two structurally different ligands”, as used herein, refers to a combination of two or more ligands of the invention as defined above, wherein said ligands differ from each other in their chemical composition in at least one position.

The ligand mixture can advantageously be organized so that the ligands bearing no sialic acid moiety provide optimal spacing for the ligands that do bear a sialic acid moiety and do not hinder the interactions between the sialic acid moieties and SARS-CoV-2, another coronavirus or an influenza virus. Thus, a ratio will exist between ligands bearing versus those not bearing a sialic acid moiety, ranging (depending upon the size of the cyclodextrin core) from at least 2 out of 6-8 ligands to 5-7 out of 6-8 ligands, preferably 3-4 out of 6-8 ligands or 4-5 out of 6-8 ligands. Also preferred are the compositions of the invention where all of the ligands (6 out of 6, 7 out of 7, and 8 out of 8) bear a sialic acid moiety.

In an embodiment, a virucidal composition of the present invention, wherein the core is cyclodextrin, is according to Formula (I)

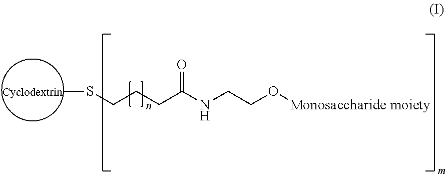

wherein
m is 2 to 8,
n is 2 to 28 or 4 to 13,
and sialic acid (SA) is the monosaccharide moiety.

Alternatively, the compositions of Formula (I) can include those wherein:
m is 2 to 8, preferably m is 3 or 4,
n is 2 to 28 (or 4 to 13 or 4 to 30 or 6 to 15, preferably 2 to 28 or 4 to 13); in some embodiments n is from 2 or 4 or 6 to 13 or 28 or 30;
and sialic acid (SA) is the monosaccharide moiety.

Preferably, the cyclodextrin of Formula (I) is selected from the group comprising alpha-cyclodextrin, beta-cyclodextrin, gamma-cyclodextrin or combinations thereof. It will be understood by those skilled in the art that the oxygen atom shown adjacent the monosaccharide moiety in Formula (I) can be considered part of the sialic acid.

Another aspect of the present invention provides a virucidal composition according to Formula (II)

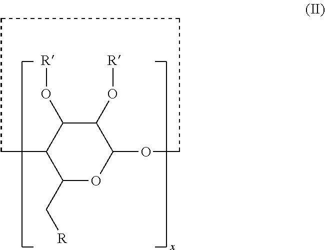

wherein:
each R, independently, is OH, SH or an optionally substituted alkyl-based ligand, wherein no more than 4 R groups can be OH or SH and at least two of said ligands have a sialic acid moiety;
each R′, independently, is H, —(CH₂)_{y}—COOH, —(CH₂)_{y}—SO⁻₃, a polymer or a water solubilizing moiety;
x is 6, 7 or 8; and
y is an integer from 4 to 20,

or a pharmaceutically acceptable salt thereof.

In one embodiment of the virucidal composition according to Formula (II), the optionally substituted alkyl-based ligands are selected from the group: alkylamidoalkoxy, alkylamidoalkylthio, carboxyalkyloxy, and carboxyalkylthio.

Alternatively, the compositions of Formula (II) can include those wherein:
each R, independently, is an optionally substituted alkyl-based ligand, wherein at least two of said ligands have a sialic acid moiety; preferably optionally substituted alkyl-based ligand is an optionally substituted C₄-C₃₀ alkyl-based ligand, more preferably an optionally substituted C₆-C₁₅ alkyl-based ligand or an optionally substituted C₆-C₁₅ alkyl-based ligand, at least two of which comprising sialic acid;
each R′, independently, is H, —(CH₂)_{y}—COOH, —(CH₂)_{y}—SO⁻₃, a polymer or a water solubilizing moiety; preferably R′ is H; preferably R′, independently, is H, —(CH₂)_{y}—COOH, —(CH₂)_{y}—SO⁻₃, or a polymer; preferably R′, independently, is —(CH₂)_{y}—COOH, —(CH₂)_{y}—SO⁻₃, or a polymer;
x is 6, 7 or 8; and
y is an integer from at least 4 to about 20, preferably y is at least 4, preferably y is 4 to 20, preferably y is 7 to 11, most preferably y is 10. In other embodiments, y is at least 6, at least 7, at least 8, at least 9, at least 10, at least 11. In other embodiments, y is at maximum 100, at maximum 70, at maximum 50, at maximum 25, at maximum 20, at maximum 15.
or a pharmaceutically acceptable salt thereof.

The ligands of virucidal compositions according to Formula (II) are as defined above and are typically sufficiently long optionally substituted alkyl-based ligands, preferably optionally substituted C₄-C₃₀ alkyl-based ligands or optionally substituted C₆-C₁₅ alkyl-based ligands, to present sialic acid (SA) for binding with a virus and are hydrophobic.

The polymer in the virucidal compositions of the invention can be selected from both synthetic and natural polymers. In an embodiment of the invention, the synthetic polymers are selected from the group comprising, but not limited to, poly(ethylene glycol) (PEG), poly(vinyl alcohol) (PVA), poly(acrylamide) (PAAm), poly(n-butyl acrylate), poly-(α-esters), (PEG-b-PPO-b-PEG), poly(N-isopropylacrylamide) (pNIPAAM), polylacticglycolic acid (PLGA) and/or combinations thereof. In another embodiment of the invention, the natural polymers are selected from the group comprising dextran, dextrins, glucose, cellulose and/or combinations thereof.

Specific virucidal compositions of the invention include “SA11” and “SA6”, both of which correspond to Formula (III) as shown below:

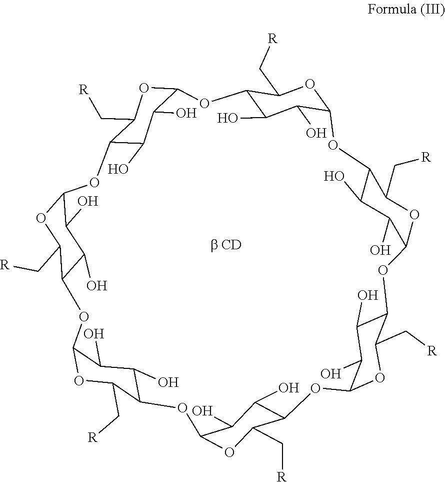

where each R group is independently selected from —OH, —SH, Formula (IV), Formula (V), Formula (VI) and Formula (VII):

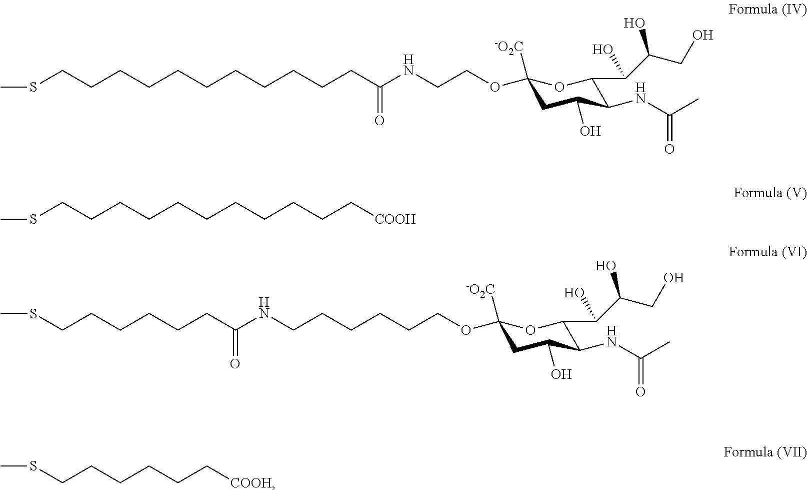

or a pharmaceutically acceptable salt thereof.

For SA11:
from 2 to 7 (especially 3 to 7, 4 to 7, or 4 to 6) R groups are represented by Formula (IV), and
from 5 to 0 (especially 4 to 0, 3 to 0, or 3 to 1) R groups are represented by Formula (V),
with 0, 1 or 2 R groups being —OH or —SH.

For SA6:
from 2 to 7 (especially 3 to 7, 4 to 7, or 4 to 6) R groups are represented by Formula (VI), and
from 5 to 0 (especially 4 to 0, 3 to 0, or 3 to 1) R groups are represented by Formula (VII),
with 0, 1 or 2 R groups being —OH or —SH.

Alternatively, the compositions of Formula (III) can include those wherein
for SA11, from 2 to 7, especially 3 to 7 R groups are represented by Formula (IV) and from 5 to 0, especially 4 to 0 R groups are represented by Formula (V):
and for SA6, from 2 to 7, especially 3 to 7 R groups are represented by Formula (VI) and from 5 to 0, especially 4 to 0 R groups are represented by Formula (VII).

Other compositions of the invention incorporating PEGylated ligands can correspond to Formula (II) or (III) where R (when not OH or SH) can be:
—O—(CH₂)_{d}—(OCH₂CH₂)ₑ—CH₂CH₂C(O)NH—(CH₂)_{f}-SA,
—S—(CH₂)_{d}—(OCH₂CH₂)ₑ—CH₂CH₂C(O)NH—(CH₂)_{f}-SA,
—O-pyrrolidine-2,5-dione-(CH₂)_{d}—(OCH₂CH₂)ₑ—CH₂CH₂C(O)NH—(CH₂)_{f}-SA, or
—S-pyrrolidine-2,5-dione-(CH₂)_{d}—(OCH₂CH₂)ₑ—CH₂CH₂C(O)NH—(CH₂)_{f}-SA,

where d is 1 to 2, e is 4 to 12, and f is 2 to 8. These include “PEGS”, a composition corresponding to Formula (III) where:
Formula (VIII) represents from 2 to 7 (especially 3 to 7, 4 to 7, or 4 to 6) R groups,

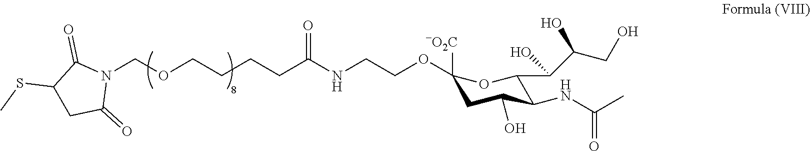

and Formula (IX) represents from 5 to 0 (especially 4 to 0, 3 to 0, or 3 to 1) R groups,

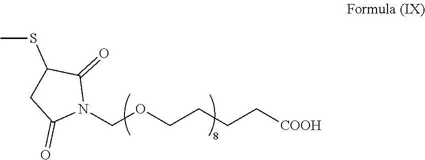

with 0, 1 or 2 R groups being —OH or —SH.

It will be appreciated by those skilled in the art that the compositions of the invention where some, but not all, of the primary face hydroxyls of cyclodextrin have been substituted by a ligand comprising a sialic acid (SA) moiety can exist as an individual isomer or as a mixture of positional isomers. Except as specifically indicated, the compositions described as synthesized and tested herein have been mixtures of such isomers; all such mixtures and single isomers being within the scope of the invention.

Particular Compositions

By way of non-limiting example, a particular group preferred for the compositions, pharmaceutical formulations, methods of manufacture and use of the present disclosure are the following combinations and permutations of substituent groups of Formulae I-III (sub-grouped, respectively, in increasing order of preference):
Formula (I) where the cyclodextrin is β-cyclodextrin and m is 3 to 7.
   Especially where n is 4 to 13.
      Particularly where n is 8 to 12.
         Preferably where m is 3 to 5.
      Particularly where n is 9 to 11.
         Preferably where m is 3 to 5.
      Particularly where n is 10.
         Preferably where m is 3 to 5.
Formula (II) where x is 7.
   Especially where R′ is H, —(CH₂)_{y}—COOH, or —(CH₂)_{y}—SO⁻₃.
   Especially where R′ is H.
      Particularly where at least 3 of the R groups are an akylamidoaklylthio ligand bearing a terminal sialic acid moiety.
         Preferably where the R groups not bearing a terminal sialic acid moiety are a C₇ to C₁₃ carboxyalkylthio ligand or SH.
         More preferably where each of the carboxyalkylthio ligands are the same.
         Preferably where the R groups not bearing a terminal sialic acid moiety are a C₉ to C₁₂ carboxyalkylthio ligand or SH.
         More preferably where each of the carboxyalkylthio ligands are the same.
         Preferably where the R groups not bearing a terminal sialic acid moiety are a C₁₀ or C₁₁ carboxyalkylthio ligand or SH.
         More preferably where each of the carboxyalkylthio ligands are the same.
      Particularly where 3 to 7 of the R groups are an akylamidoaklylthio ligand bearing a terminal sialic acid moiety.
         Preferably where the R groups bearing a terminal sialic acid moiety are C₂-to-C₆-alkylamido-C₆-to-C₁₁-alkylthio ligands.
         More preferably where the R groups not bearing a terminal sialic acid moiety are a C₇ to C₁₃ carboxyalkylthio ligand or SH.
         Still more preferably where each of the carboxyalkylthio ligands are the same.
         More preferably where the R groups not bearing a terminal sialic acid moiety are a C₉ to C₁₂ carboxyalkylthio ligand or SH.
         Still more preferably where each of the carboxyalkylthio ligands are the same.
         More preferably where the R groups not bearing a terminal sialic acid moiety are a C₁₀ or C₁₁ carboxyalkylthio ligand. or SH
         Still more preferably where each of the carboxyalkylthio ligands are the same.
         Preferably where the R groups not bearing a terminal sialic acid moiety are a C₇ to C₁₃ carboxyalkylthio ligand or SH.
         More preferably where each of the carboxyalkylthio ligands are the same.
         Preferably where the R groups not bearing a terminal sialic acid moiety are a C₉ to C₁₂ carboxyalkylthio ligand or SH.
         More preferably where each of the carboxyalkylthio ligands are the same.
         Preferably where the R groups not bearing a terminal sialic acid moiety are a C₁₀ or C₁₁ carboxyalkylthio ligand or SH.
         More preferably where each of the carboxyalkylthio ligands are the same.
Formula (III) where 3 to 7 R groups are represented by Formula (IV) and 4 to 0 R groups are represented by Formula (V).
   Especially where 3 to 6 R groups are represented by Formula (IV).
      Preferably where 4 to 5 R groups are represented by Formula (IV).
      Preferably where 5 to 6 R groups are represented by Formula (IV).
Formula (III) where 3 to 7 R groups are represented by Formula (VI) and 4 to 0 R groups are represented by Formula (VII).
   Especially where 3 to 6 R groups are represented by Formula (VI).
      Preferably where 4 to 5 R groups are represented by Formula (VI).
      Preferably where 5 to 6 R groups are represented by Formula (VI).

### Preparation of the Compositions of the Invention

### Synthetic Reaction Parameters

The terms “solvent”, “inert organic solvent” or “inert solvent” mean a solvent inert under the conditions of the reaction being described in conjunction therewith [including, for example, benzene, toluene, acetonitrile, tetrahydrofuran (“THF”), dimethylformamide (“DMF”), dimethylsulfoxide (“DMSO”), chloroform, methylene chloride (or dichloromethane), diethyl ether, methanol, pyridine and the like]. Unless specified to the contrary, the solvents used in the reactions of the present invention are inert organic solvents.

Isolation and purification of the compounds and intermediates described herein can be effected, if desired, by any suitable separation or purification procedure such as, for example, filtration, extraction, crystallization, column chromatography, thin-layer chromatography or thick-layer chromatography, centrifugal size exclusion chromatography, high-performance liquid chromatography, recrystallization, sublimation, fast protein liquid chromatography, gel electrophoresis, dialysis, or a combination of these procedures. Specific illustrations of suitable separation and isolation procedures can be had by reference to the examples hereinbelow. However, other equivalent separation or isolation procedures can, of course, also be used.

Unless otherwise specified (including in the examples), all reactions are conducted at standard atmospheric pressure (about 1 atmosphere) and ambient (or room) temperature (ranging from about 18° C. to 30° C., most typically about 20° C.), at about pH 7.0-8.0.

Characterization of reaction products can be made by customary means, e.g., proton and carbon NMR, mass spectrometry, size exclusion chromatography, infrared spectroscopy, gel electrophoresis.

The compositions of the invention can be prepared, for example, as described in WO 2020/048976, substituting Neu5Acα(2,6)-Galβ(1-4)-GlcNAc-β-ethylamine with 5-acetamido-2-O-(2-aminoethyl)-3,5-dideoxy-D-glycero-D-galacto-2-nonulo-pyranosidonic acid or 5-acetamido-2-O-(6-aminohexyl)-3,5-dideoxy-D-glycero-D-galacto-2-nonulopyranosidonic acid in the Example “Synthesis of Modified Cyclodextrins, Step 3: Trisaccharide grafting”. The aminoalkyl sialic acid reactant employed in synthesizing the compositions of the invention can be prepared as described in the publication Šardzík et al., Preparation of aminoethyl glycosides for glycoconjugation, Beilstein J. Org. Chem. 2010, 699-703. Alternative syntheses of the compositions of the invention are described below with reference to Reaction Schemes 1-2.

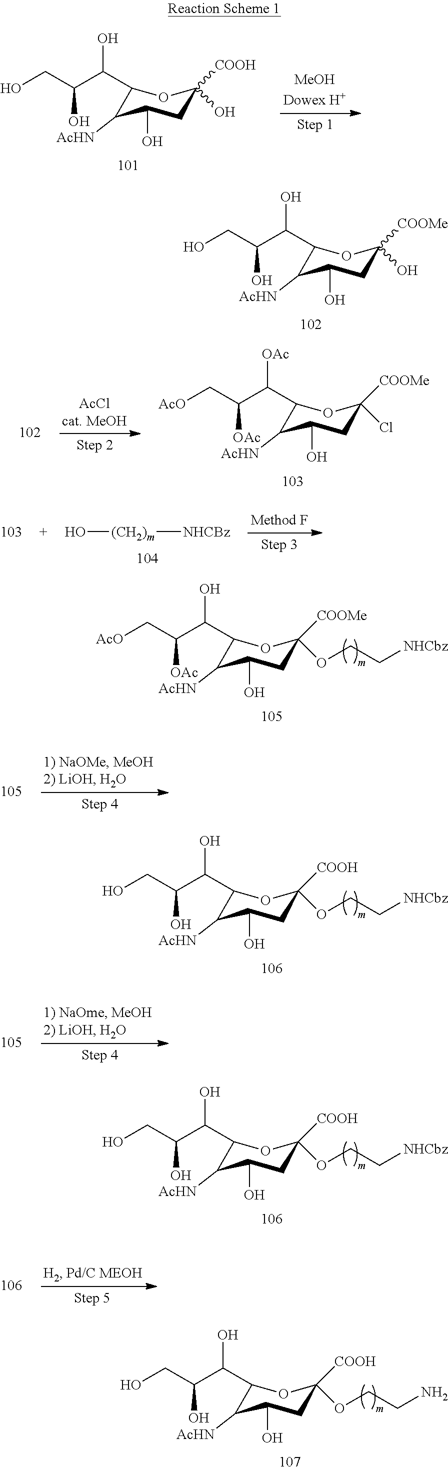

### Preparation of Aminoalkyl Sialic Acid Reactant

### Preparation of Formula 102

Referring to Reaction Scheme 1, Step 1, N-Acetyl neuraminic acid (101) (Codexis) is stirred with Dowex 50WX4 in a suitable solvent, e.g., dry methanol. The reaction takes place over a period of 3 to 25 hours, preferably 6 to 18 hours, and most preferably about 12 hours. The removal of the resin by filtration and evaporation of the solvent in vacuo affords the methyl ester of Formula 102, N-Acetyl β-neuraminic acid methyl ester, as a white solid.

### Preparation of Formula 103

Referring to Reaction Scheme 1, Step 2, the methyl ester of Formula 102 is dissolved in acetyl chloride and abs methanol is added. The reaction vessel is sealed and the mixture stirred. The reaction takes place over a period of 3 to 7 days preferably 5 days. Evaporation to dryness following by short column chromatography on silica (EtOAc/hexane 80/20) gives the chloride of Formula 103, Methyl 5-acetamido-4,7,8,9-tetra-O-acetyl-2,3,5-trideoxy-2-chloro-D-glycero D-galacto-2-nonulopyranosonate, as a yellowish solid. Recrystallisation from diethyl ether/petroleum ether gave the title compound as a white crystalline solid.

### Preparation of Formula 105

Referring to Reaction Scheme 1, Step 3, the sialyl chloride of Formula 103 and about 2.5 molar equivalents of a N-Cbz-aminoalkanol of Formula 104, where m can be 2 to 8, are dissolved in a suitable solvent, e.g., CH₂Cl₂ and a suitable amount of 4 Å molecular sieves is added. After stirring for about 1 h, Ag₂CO₃ (about 2 equivalents) is added and the mixture stirred. The reaction takes place over a period of 12 top 24 hours, preferably 16 hours, with exclusion of light. The resulting solid is filtered (e.g., through Celite), washed (e.g., with CH₂Cl₂) and the filtrate evaporated to dryness. Column chromatography (e.g., EtOAc/hexane 80:20) gives the glycoside of Formula 105.

### Preparation of Formula 106

Referring to Reaction Scheme 1, Step 4, the aminoalkyl glycoside of Formula 105 is dissolved in in a suitable solvent, e.g., methanol, and about 1 molar equivalent of sodium methoxide, dissolved a suitable solvent, e.g., methanol, is added with stirring. The solution is neutralised with Dowex 50WX8-100 (H⁺) resin, filtered and concentrated in vacuo. The reaction takes place over a period of 3 to 10 hours, preferably 6 hours. The resulting residue is dissolved in a suitable solvent, e.g., water/methanol (4:1), treated with LiOH (about 3 equivalents) and the solution is stirred, e.g., overnight at room temperature. After neutralisation with Dowex 50WX8-100, the resin is filtered off and washed, e.g., with methanol/water (1:1). The filtrate is concentrated in vacuum to remove most of the solvent system and then freeze dried to yield the corresponding free glycoside of Formula 106.

### Preparation of Formula 107

Referring to Reaction Scheme 1, Step 5, glycoside of Formula 106 is hydrogenated, e.g., in methanol (5 mL) with palladium on carbon. The hydrogenation takes place over a period of 3 to 10 hours, preferably 6 hours. The hydrogenated product is isolated and purified. For example, it can be filtered through Celite to remove the catalyst, the filter cake washed with methanol and the filtrates concentrated in vacuo. The product can then be dissolved in water, treated with activated charcoal and filtered. Lyophilisation of the filtrate gives the deprotected sialic acid of Formula 107. This sialic acid's carboxylic acid can, depending on reaction parameters such as solvent system, be produced as a free acid or a pharmaceutically acceptable salt.

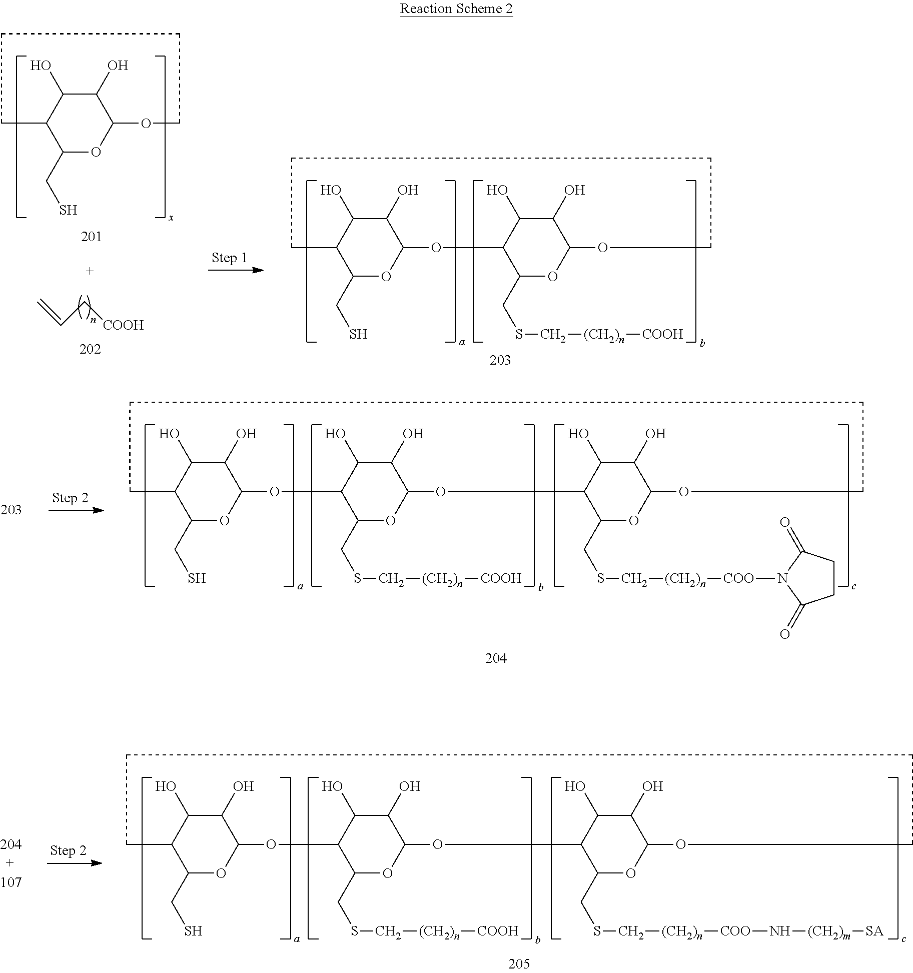

### Preparation of Formula 203

Referring to Reaction Scheme 2, Step 1, a mercapto-modified cyclodextrin such as Formula 201 where x is 6, 7 or 8 is contacted with about 6-8 molar equivalents of a bi-functional molecule bearing an allyl and a carboxylic acid group illustrated as Formula 202, where n is 4 to 20, in a suitable solvent such as DMSO or DMF, and is subjected to a photochemical reaction (UV light or a dedicated photoreactor). Alternatively, halogenated cyclodextrine and carboxyalkylthiol starting materials can be employed under basic conditions to eliminate the need to use UV light. The reaction takes place over a period of 3 to 25 hours, preferably 6 to 18 hours, and most preferably about 12 hours depending on the scale. The intermediate of Formula 203, where a is 0 to 4 and b is 3 to 8 (and a+b=x), is carried forward without isolation or further purification.

### Preparation of Formula 204

Referring to Reaction Scheme 2, Step 2, to the modified cyclodextrin of Formula 203 is added a significant (e.g., 4-5 molar equivalents) of NHS plus EDC-HCl and DMAP. The reaction takes place over a period of 3 to 25 hours, preferably 6 to 18 hours, and most preferably about 12 hours. The intermediate of Formula 204, where a is 0 to 4 and b is 0 to 4 and c is 3 to 8 (and a+b+c=x), is purified before further use, e.g., by precipitation and centrifugation. Other amide couplers such as DMTMM can be used in this step. Ideally, it is recommended to activated the carboxylates and remove amide couplers to avoid formation of dimers, trimer and oligomers of the sialic acid that contains amine and carboxy functionalities.

### Sialic Acid Grafting

Referring to Reaction Scheme 2, Step 3, a sialic acid alkyl amine of Formula 107, prepared, e.g., as illustrated with in Reaction Scheme 1, is contacted with the cyclodextrin derivative protected by NHS groups for amide bond formation in the presence of 0.5 to 1.0 molar equivalents of TEA (triethylamine) and in a suitable solvent such as DMSO or DMF (aqueous amide couplings are possible). The reaction takes place over a period of 12 to 24 hours, preferably 12 to 18 hours, and most preferably about 12 hours. Under aqueous conditions, the reaction is faster and can be complete in 2 to 4 hours. The product of Formula 205, where a is 0 to 4 and b is 0 to 4 and c is 3 to 8 (and a+b+c=x), is isolated and purified by repeated precipitations and decantations: washing, centrifuging, filtering and dialysis followed by lyophilization to isolate a colorless product. Formula 205 corresponds to a virucidal composition of the invention within the scope of Formulae (I), (II) and (III).

### Preparation of PEGylated Compositions

The compositions of the invention that employ PEGylated ligands can be prepared, for example, by substituting an equivalent amount of an allyl-polyethoxy carboxylic acid (e.g., propargyl-PEG6-acid) for Formula 202 and following through from Reaction Scheme 2, Step 1. Alternatively, a 1-(2,5-dioxo-2,5-dihydro-1H-pyrrol-1-yl)-polyethoxy alkanoic acid (2 molar equivalents for each R group) can be substituted for Formula 202 using a polar solvent (e.g., DMSO or DMF) in Reaction Scheme 2, Step 1, proceeding without exposure to UV light and allowing the reaction to proceed for several days to afford the PEGylated pyrrolidine-2,5-dione thio corresponding to Formula 203 and following through from Reaction Scheme 2, Step 2.

Particular Processes and Last Steps

An NHS (or other amide coupling) activated modified cyclodextrin is contacted with a sialyl acid alkyl amine under basic conditions.

Utility, Testing and Administration

General Utility

An aspect of the invention provides a method of treating and/or preventing COVID-19 and other respiratory diseases caused by coronaviruses, influenza virus infections and/or diseases associated therewith, comprising administering to a subject in need thereof, a therapeutically effective amount of one or more virucidal compositions of the invention. These treatable viruses and diseases are discussed in greater detail below.

Coronaviruses (CoVs) are abundant and tend to cause mild to serious upper-respiratory tract syndromes, like the common cold or lower respiratory diseases like wheezy bronchitis and other affections of the lower respiratory tract. Coronaviruses tend to reinfect the same human hosts (Archives of Disease in Childhood, 1983, 58, 500-503). Coronaviruses are zoonotic and circulate among pigs, horses, cats, bats, camels, among other species. When a coronavirus jumps from an animal to humans, they can cause the mild to moderate diseases associated to coronaviruses such as HCV229E (alpha CoV), HCVOC43 (beta CoV), HCVNL63 (alpha CoV), HCVOC43 (beta coronavirus), HCVHKU1 (beta coronavirus), all of which do not have a distinct pathognomonic syndrome named after the individual virus. In the last two decades, three CoVs have caused serious respiratory (upper and lower) syndromes with dedicated syndromes being named to describe their infections: MERS-CoV (beta CoV that causes Middle East Respiratory Syndrome, or MERS); SARS-CoV (the beta CoV that causes severe acute respiratory syndrome, or SARS) and SARS-CoV-2 (the novel CoV that causes COVID-19). Like all viruses, CoVs use either Sialic Acids (SAs) and/or Heparan Sulfate Proteoglycans (HSPGs), among other cell surface receptors such as the Angiotensin Converting Enzyme to infect the host cell. HCVNL63 and SARS-CoV use primarily SAs to dock onto host cells while the docking used by other variants is still under investigation (Microorganisms 2020, 8, 1894; doi:10.3390/microorganisms8121894).

CoVs are also of great importance in the veterinary and livestock industries because they cause diseases to animals. The Equine Coronavirus (ECoV), a beta-CoV causes enteric inflammation on horses and is closely related to the bovine CoV (BCoV), also a beta-CoV that causes enzootic pneumonia complex and dysentery in calves and has been reported to cause winter dysentery in adult cattle. Both ECoV and BCoV infect the host cells via the N-acetyl-9-O-acetylneuraminic acid receptor, also referred to as Sialic acid. In pigs, the Porcine Respiratory Coronavirus (PRCv) causes a respiratory disease to which the only treatment is isolation of the contaminated animal. Other CoVs affect pigs, such as Transmissible Gastroenteritis Virus (TGEV), Porcine epidemic diarrhoea virus (PEDV), and porcine haemagglutinating encephalomyelitis virus (PHEV). PDCoV (porcine deltacoronavirus) TGEV and PRCV are alpha CoVs and closely associated to the CoVs that affect cats and dogs, and to PEDV and human CoVs HCV229E and HCVNL63. PHEV and PDCoV are the beta CoVs. Poultry and many avian species also develop diseases caused by CoVs such as coronaviruses of the domestic fowl—infectious bronchitis virus IBV, that causes respiratory illness to chicken (Gallus gallus), turkey (Meleagris gallopavo) and pheasant (Phasianus colchicus). Improvements in testing and detection will likely increase the list of coronaviruses that affect animals. The fear of new outbreaks of CoVs relevant to human health may also increase this list as the source of new outbreaks lies predominantly in livestock.

Influenza viruses are sialic acid dependent viruses that cause a syndrome referred to as the flu. Four types of influenza viruses, A, B, C and D, affect humans. Human influenza A and B cause seasonal flu syndrome epidemics almost every winter, alternating the winters of the northern and southern hemispheres. Influenza A are the only viruses known to cause flu pandemics (global epidemics) of the flu. A new variant of influenza A virus can infect people and spread rapidly. Influenza C infections generally cause mild illness and are not thought to cause human flu epidemics. Influenza D viruses primarily affect cattle and are not known to infect or cause illness in people.

Influenza A viruses are divided into subtypes based on two proteins on the surface of the virus: hemagglutinin (H) and neuraminidase (N). There are 18 different hemagglutinin subtypes and 11 different neuraminidase subtypes (H1 through H18 and N1 through N11, respectively). While there are potentially 198 different influenza A subtype combinations, only 131 subtypes have been detected in nature. Current subtypes of influenza A viruses that routinely circulate in people include: A(H1N1) and A(H3N2). Influenza A subtypes can be further broken down into different genetic “clades” and “sub-clades.” Therefore, flus caused by H(x)N(y) where x and y refer to the subtype of H and N can be tackled with a specifically designed Sialic Acid mimic such as SA11 to treat flus in humans and in animals, particularly including livestock (e.g., avian, porcine and others).

As shown in Examples, the virucidal compositions of the invention surprisingly showed efficacy in treatment of COVID-19. Further, it has surprisingly been discovered that influenza virus does not develop resistance against the virucidal compositions of the invention.

Another aspect of the invention provides the virucidal compositions of the invention for use in treating and/or preventing COVID-19, influenza virus infections and/or diseases associated therewith.

Another aspect of the invention provides a method of disinfection and/or sterilization using the virucidal compositions of the invention or the virucidal composition of the invention or the pharmaceutical composition of the invention.

In a preferred embodiment, the method of disinfection and/or sterilization comprises the steps of (i) providing at least one virucidal composition of the invention or a virucidal composition of the invention, or pharmaceutical composition of the invention, (ii) contacting a viruses-contaminated surface or a surface suspected to be contaminated by a virus with the at least one virucidal composition of the invention or a virucidal composition of the invention or pharmaceutical composition of the invention for a time sufficient to obtain virucidal effect. In some embodiments, the virus contaminated surface is human or animal skin. In other embodiments, the virus contaminated surface is a non-living surface, such as medical equipments, clothing, masks, furnitures, rooms, etc.

Another aspect of the invention provides a use of a virucidal composition of the invention or a virucidal composition of the invention or a pharmaceutical composition of the invention for sterilization and/or for disinfection. In some embodiments, sterilization and disinfection is for viruses-contaminated surfaces or surfaces suspected to be contaminated by viruses. In some preferred embodiments, the surfaces are human or animal skin. In other preferred embodiments, the surfaces are non-living surfaces, such as medical equipments, clothing, masks, furnitures, rooms, etc. In an embodiment, the virucidal composition of the invention or the pharmaceutical composition of the invention is used as virucidal hand disinfectant for frequent use. In another embodiment, the virucidal composition of the invention or the pharmaceutical composition of the invention is applied by spraying. In a further embodiment, the virucidal composition of the invention of the pharmaceutical composition of the invention is applied on a protective mask.

Testing

In vitro activity for SARS-CoV-2 Inhibition is determined, for example, as described in Gasbarri et al., Microorganisms 2020, 8, 1894 (2020).

In vitro and in vivo activity for influenza can be determined, for example, as described in Kocabiyik et al., Non-Toxic Virucidal Macromolecules Show High Efficacy Against Influenza Virus Ex Vivo and In Vivo, Adv. Sci. 2020, 2001012 (DOI: 10.1002/advs.202001012).

Administration

Dosage

The amount of a virucidal composition of the invention that can be combined with a carrier material to produce a single dosage form will vary depending upon the viral disease treated, the mammalian species, and the particular mode of administration. It will be also understood, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular viral disease undergoing therapy, as is well understood by those of skill in the area.

While human dosage levels have yet to be optimized for the compositions of the invention, generally, a daily inhaled dose is from about 0.01 to 50.0 mg/kg of body weight, preferably about 0.1 to 20.0 mg/kg of body weight, and most preferably about 3.0 to 13.0 mg/kg of body weight. Thus, for administration to a 70 kg person, the dosage range would be about 0.7 to 3,500.0 mg per day, preferably about 7.0 to 1,400.0 mg per day, and most preferably about 210.0 to 910.0 mg per day.

Formulation

An aspect of the invention discloses a pharmaceutical composition comprising an effective amount of one or more virucidal compositions of the invention and at least one pharmaceutically acceptable excipient, carrier and/or diluent. Optionally, the pharmaceutical composition of the present invention further comprises one or more additional active agents, preferably anti-viral agents.

As to the appropriate excipients, carriers and diluents, reference may be made to the standard literature describing these, e.g. to chapter 25.2 of Vol. 5 of “Comprehensive Medicinal Chemistry”, Pergamon Press 1990, and to “Lexikon der Hilfsstoffe für Pharmazie, Kosmetik and angrenzende Gebiete”, by H. P. Fiedler, Editio Cantor, 2002. The term “pharmaceutically acceptable carrier, excipient and/or diluent” means a carrier, excipient or diluent that is useful in preparing a pharmaceutical composition that is generally safe, and possesses acceptable toxicities. Acceptable carriers, excipients or diluents include those that are acceptable for veterinary use as well as human pharmaceutical use. A “pharmaceutically acceptable carrier, excipient and/or diluent” as used in the specification and claims includes both one and more than one such carrier, excipient and/or diluent.

The virucidal compositions of the invention that are used in the methods of the present invention can be incorporated into a variety of formulations and medicaments for therapeutic administration. More particularly, a virucidal composition as provided herein can be formulated into pharmaceutical compositions by combination with appropriate, pharmaceutically acceptable carriers, excipients and/or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of the virucidal compositions can be achieved in various ways, including oral, buccal, inhalation (pulmonary, nasal), rectal, parenteral, intraperitoneal, intradermal, transdermal, intracranial and/or intratracheal administration. Moreover, the virucidal compositions can be administered in a local rather than systemic manner, in a depot or sustained release formulation. The virucidal compositions can be formulated with common excipients, diluents or carriers, and compressed into tablets, or formulated as elixirs or solutions for convenient oral administration, or administered by the intramuscular or intravenous routes. The virucidal compositions can be administered transdermally and can be formulated as sustained release dosage forms and the like. The virucidal compositions can be administered alone, in combination with each other, or they can be used in combination with other known compounds. Suitable formulations for use in the present invention are found in Remington's Pharmaceutical Sciences (Mack Publishing Company (1985) Philadelphia, Pa., 17th ed.), which is incorporated herein by reference. Moreover, for a brief review of methods for drug delivery, see, Langer, Science (1990) 249:1527-1533, which is incorporated herein by reference.

Sustained-release preparations may be prepared. Suitable examples of sustained-release preparations include semi permeable matrices of solid hydrophobic polymers containing the virucidal compositions of the invention, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and [gamma] ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The virucidal compositions of the present invention may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting. For injection, a virucidal composition (and optionally another active agent) can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, the virucidal compositions of the present invention can be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Preferably, pharmaceutical formulations for parenteral administration include aqueous solutions of the virucidal compositions in water-soluble form. Additionally, suspensions of the virucidal compositions can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the virucidal compositions to allow for the preparation of highly concentrated solutions.

Formulations of the active composition or a salt can also be administered to the respiratory tract as an aerosol or solution for a nebulizer (including a propellant), or as a microfine powder for insufflation, alone or in combination with an inert carrier/excipient such as lactose, trehalose dextrin, mannitol and leukin inulin. In such a case, the particles of the formulation have diameters of less than 50 microns, preferably less than 10 microns.

Additional Products

Another aspect of the invention provides a virucidal composition comprising an effective amount of one or more virucidal compositions of the invention and optionally at least one suitable carrier or aerosol carrier. “An effective amount” refers to the amount sufficient for irreversibly inhibiting SARS-CoV-2, another coronavirus or influenza viruses; i.e. sufficient for obtaining virucidal effect. In an embodiment, the suitable carrier is selected from the group comprising stabilisers, fragrance, colorants, emulsifiers, thickeners, wetting agents, or mixtures thereof. In another embodiment, the virucidal composition can be in the form of a liquid, a gel, a foam, a spray or an emulsion. In a further embodiment, the virucidal composition can be an air freshener, a sterilizing solution or a disinfecting solution.

Another aspect of the invention provides a device (or a product) for disinfection and/or sterilization comprising the virucidal composition of the invention or one or more virucidal compositions of the invention and means for applying and/or dispensing the virucidal compositions of the invention. In another embodiment, the means comprise a dispenser, a spray applicator or a solid support soaked with the virucidal compositions of the invention. In another embodiment, the support is a woven or non-woven fabric, a textile, a paper towel, cotton wool, an absorbent polymer sheet, or a sponge.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

The foregoing description will be more fully understood with reference to the following Examples. Such Examples, are, however, exemplary of methods of practising the present invention and are not intended to limit the scope of the invention. Numbers shown in bold, e.g., 102, refer to the correspondingly identified structures in the reaction schemes.

### EXAMPLES

### Example 1

### Synthesis of SA11

### A. N-Acetyl β-Neuraminic Acid Methyl Ester (102)

N-Acetyl neuraminic acid (Codexis, 2.00 g, 6.47 mmol) was stirred with Dowex 50WX4 (500 mg) in dry methanol (150 mL) overnight at r.t. The removal of the resin by filtration and evaporation of MeOH in vacuo afforded methyl ester 102 (2.0 g, 6.19 mmol, 96%) as a white solid.

### B. Methyl 5-acetamido-4,7,8,9-tetra-O-acetyl-2,3,5-trideoxy-2-chloro-D-glycero-β-D-galacto-2-nonulopyranosonate (103)

Methyl ester 102 (2.00 g, 6.19 mmol) was evaporated with toluene for three times to remove water residue and then dissolved in acetyl chloride (60 mL) and abs methanol (1.2 mL) added. The reaction vessel was sealed and the mixture stirred at r.t. for 2 days. The mixture was evaporated to dryness with toluene for three times and dissolved in 2 mL toluene, added dropwise to 100 mL hexane with the most violent stirring. The white precipitation (2.28 g, 4.47 mmol, 72%) was filtered and collected after one hour.

### C. Methyl 5-acetamido-2-O-(2-benzyloxycarbonylaminoethyl)-4,7,8,9-tetra-O-acetyl-3,5-dideoxy-D-glycero-α-D-galacto-2-nonulopyranosonate (105)

Referred to as “Method F” in Reaction Scheme 1. Sialyl chloride 103 (400 mg, 0.784 mmol) and N-Cbz-aminoethanol (383 mg, 1.96 mmol, 2.5 equiv) were dissolved in dry CH₂Cl₂ (6 mL) and 500 mg of 4 Å molecular sieves was added. After stirring for 1 h, Ag₂CO₃ (433 mg, 1.57 mmol, 2 equiv) was added and the mixture stirred for 16 h at room temperature with exclusion of light. The solid was filtered through Celite, washed with CH₂Cl₂ and the filtrate evaporated to dryness. Column chromatography (gradient eluent: hexane to EtOAc/hexane 80:20) gave 368 mg (0.55 mmol, 70%) of glycoside 105, where a is 2, as a colourless foam.

### D. 5-Acetamido-2-O-(2-benzyloxycarbonylaminoethyl)-3,5-dideoxy-D-glycero-D-galacto-2-nonulopyranosidonic acid (106)

Aminoethyl glycoside 105 (80 mg, 0.12 mmol) was dissolved in methanol (4 mL) and sodium methoxide was added to adjust the pH to 9-10. When the starting material disappeared on TLC (EtOAc/hexane 80:20), the solution was neutralised with Dowex 50WX8-100 (H+) resin, filtered and concentrated in vacuo. The residue (Rf=0.4, DCM:MeOH=5:1) was dissolved in 3 mL water/methanol (4:1), treated with LiOH (8.6 mg, 0.36 mmol) and the solution was stirred overnight at room temperature. After neutralisation with Dowex 50WX8-100, the resin was filtered off and washed with methanol/water (1:1). The filtrate was concentrated in vacuum to remove most of the methanol and then freeze dried to yield the free glycoside 106, where a is 2, as a white solid (52 mg, 0.11 mmol, 89%, Rf=0.3, CHCl₃:MeOH=3:2).

### E. 5-Acetamido-2-O-(2-aminoethyl)-3,5-dideoxy-D-glycero-α-D-galacto-2-nonulopyranosidonic acid (107)

The sialic acid 106 (52 mg, 0.11 mmol) was hydrogenated in methanol (5 mL) with 52 mg palladium on carbon (10%). After 6 h solution was filtered to remove the catalyst, the filter cake washed with methanol and the filtrates concentrated in vacuo. The product was redissolved in water, treated with activated charcoal and filtered. Lyophilisation of the filtrate gave the deprotected sialic acid alkyl amine 107, where a is 2 (36 mg, 0.10 mmol, 96%) as a white solid.

### F. Formula 203

Heptakis-(6-deoxy-6-mercapto)-beta-Cyclodextrin 0.02 mmol (25 mg) was mixed with 0.14 mmol (27 mg) of 11-dodecenoic acid in 2.5 mL of DMSO. An overnight reaction was conducted under the UV light to afford the product of Formula 203 where n is 10, a is 0-4, and b is 3-8, which is carried forward for the synthesis of SA11.

### G. Formula 204

To the reaction mixture described above, 40 mg of NHS (4-5 eq), 30 mg of EDC-HCl (weigh quickly) and 1 mg of DMAP was added. The activation reaction was conducted overnight to afford the corresponding product of Formula 204 where n is 10, a is 0-4, b is 0-3 and c is 3-8.

The reaction solution obtained above was transferred to a 50 mL falcon tube and purified next day, as follows:

1. Add 20 mL of cold acidic water (10 uL of HCl (1M)+200 mL of MQ water). Centrifuge the precipitate (1 min at 5000 rpm), discard liquid. Add 2 mL of DMSO and dissolve, then add 18 mL cold acidic water to precipitate the product. Centrifuge the precipitate (1 min, 5000 rpm), discard liquid. Repeat 3 times.

2. Add 10 mL of acetonitrile. Centrifuge (1 min, 5000 rpm), discard liquid.

3. Add 10 mL of Et₂O. Centrifuge (1 min, 5000 rpm), discard liquid. Dry under vacuum (1-2 h).

### H. SA11

Sialic acid ethylamine 107 (4.4 mg-8.8 mg) (note: range of equivalents reflects that SA-ethylamine purity differs from batch to batch), obtained as described in Example 1E, was mixed with 5 mg of the CD derivative of Formula 204, obtained as described in Example 1F. To this is added 50 μL of a TEA solution (25 mg of TEA+500 μL of DMSO) plus 950 μL DMSO (q.s. to make sure the reaction volume is 1 mL) and the reaction was conducted overnight at room temperature to afford the product of Formula 205 that is SA11. The product was purified as follows:

1. Wash an amicon filter (MWCO:3k) with MQ water once.

2. Dilute the product solution in 20 ml of MQ water.

3. Transfer the solution (around 10 mL for one time) to the filter and centrifuge for 20 min at 5000 rpm (10 mL each run).

4. Add 10 mL of 0.01M phosphate buffer (pH=7.5 (7.4-7.6), filtered through 0.2 um syringe filter) and centrifuge (5000 rpm, 20 min).

5. Wash with 10 mL MQ water and centrifuge (5000 rpm, 20 min) twice.

6. Wash the product of the filter with 10 mL of MQ water and transfer it to a 15 mL falcon tube.

7. Dialyze the crude product against MQ water using 1 kDa dialysis bag for three days, change the water every day.

8. After dialysis transfer the solution on amicon filter (MWCO:3k), then wash with water two times.

9. Wash CD of the filter with 2 mL of MQ water to a 15 mL (should be 2-3 mL) falcon tube, filter with 0.22 μm hydrophilic membrane and freeze dry.

### Example 2

### Synthesis of SA6

### A. Formula 203

The procedure of Example 1F was followed, substituting 11-dodecenoic acid with 0.14 mmol of 6-hexanoic acid in 2.5 mL of DMSO to afford the corresponding product of Formula 203 where n is 5.

### B. Formula 204

The procedure of Example 1G was followed, substituting the product of Formula 203 obtained in Example 2A to afford the corresponding product of Formula 204 where n is 5.

### C. Formula 107

The procedure of Example 1C was followed, substituting 6-(Z-amino)-1-hexanol for 6-(Z-amino)-1-ethanol to afford the corresponding product of Formula 107 where a is 6.

### D. SA6

The procedure of Example 1H was followed, substituting the reactants obtained in Example 2B and 2C to afford SA6.

### Example 3

### Synthesis of PEG8

### A. PEGylated β-Cyclodextrin

0.04 mmol Heptakis-(6-deoxy-6-mercapto)-beta-cyclodextrin is stirred with 0.28 mmol of maleimide-PEG₈-CH₂CH₂COOH in 1 mL of DMSO for 48 hours. The modified β-cyclodextrin is diluted into 35 mL of MilliQ water and dialysed against MilliQ water for 3 days using 1 kDA MWCO regenerated cellulose membranes. The solution is freeze-dried and the pegylated product isolated as a yellow waxy material.

### B. NHS Activation

To the product obtained in Example 3A, 40 mg of NHS (4-5 eq), 30 mg of EDC-HCl and 1 mg of DMAP are added in 1 mL of DSMO. The activation reaction is conducted overnight to afford the activated NHS ester of the pegylated product, which is purified as follows:

1. Add 20 mL of cold acidic water (10 uL of HCl (1M)+200 mL of MQ water). Centrifuge the precipitate (1 min at 5000 rpm), discard liquid. Add 2 mL of DMSO and dissolve, then add 18 mL cold acidic water to precipitate the product. Centrifuge the precipitate (1 min, 5000 rpm), discard liquid. Repeat 3 times.

2. Add 10 mL of acetonitrile. Centrifuge (1 min, 5000 rpm), discard liquid.

3. Add 10 mL of Et₂O. Centrifuge (1 min, 5000 rpm), discard liquid. Dry under vacuum (1-2 h).

### C. PEG8

Sialic acid ethylamine 107 (4.4 mg-8.8 mg), obtained, for example, as described in Example 1E, is mixed with 5 mg of the NHS-activated CD derivative obtained, for example, as described in Example 2B, 15 μmol (2.5 mg). To this is added 50 μL of a TEA solution (25 mg of TEA+500 μL of DMSO) plus 950 μL DMSO and the reaction is conducted overnight at room temperature to afford the product, PEG8. The product is purified as follows:

1. Wash an amicon filter (MWCO:3k) with MQ water once.

2. Dilute the product solution in 20 ml of MQ water.

3. Transfer the solution (around 10 mL for one time) to the filter and centrifuge for 20 min at 5000 rpm (10 mL each run).

4. Add 10 mL of 0.01M phosphate buffer (pH=7.5 (7.4-7.6), filtered through 0.2 μm syringe filter) and centrifuge (5000 rpm, 20 min).

5. Wash with 10 mL MQ water and centrifuge (5000 rpm, 20 min) twice.

6. Wash the product of the filter with 10 mL of MQ water and transfer it to a 15 mL falcon tube.

7. Dialyze the crude product against MQ water using 1 kDa dialysis bag for three days, change the water every day.

8. After dialysis transfer the solution on amicon filter (MWCO:3k), then wash with water two times.

9. Wash CD of the filter with 2 mL of MQ water to a 15 mL (should be 2-3 mL) falcon tube, filter with 0.22 μm hydrophilic membrane and freeze dry.

### Example 4

### Inhibition of SARS-CoV-2

### A. Cells and Virus

Vero C1008 (clone E6) (ATCC CRL-1586) cells are propagated in DMEM High Glucose+Glutamax supplemented with 10% fetal bovine serum (FBS) and 1% penicillin/streptavidin (pen/strep).

SARS-CoV2/Switzerland/GE9586/2020 is isolated from a clinical specimen in Vero-E6 and passaged twice before the experiments. SARS-CoV-2/München-1.1/2020/929 is propagated on Vero-E6 cells cultured in Dulbecco's modified minimal essential medium supplemented with 10% heat inactivated fetal bovine serum, 1% non-essential amino acids, 100 μg/mL of streptomycin, 100 IU/mL of penicillin, and 15 mM of HEPES. Supernatant of infected cells is collected 3 days post infection, clarified, aliquoted, and frozen at −80° C. and subsequently titrated by plaque assay in Vero-E6.

### B. VSV-CoV-2 Production

Vesicular stomatitis virus (VSV)-based SARS-CoV-2 pseudotypes (VSV-CoV-2) generated according to Berger Rentsch, M.; Zimmer, G. (A vesicular stomatitis virus replicon-based bioassay for the rapid and sensitive determination of multi-species type I interferon. PLoS ONE 2011, 6, e25858) and Fukushi, S.; Mizutani, T.; Saijo, M.; Matsuyama, S.; Miyajima, N.; Taguchi, F.; Itamura, S.; Kurane, I.; Morikawa, S. (Vesicular stomatitis virus pseudotyped with severe acute respiratory syndrome coronavirus spike protein. J. Gen. Virol. 2005, 86, 2269-2274) expressing a 19 amino acids C-terminal truncated spike protein (NCBI Reference sequence: NC_045512.2) are produced in HEK293F and titrated in Vero-E6.

### C. VSV-CoV-2 Inhibition Assays

Vero-E6 cells (13,000 cells per well) are seeded in a 96-well plate. Test compounds are serially diluted in DMEM and incubated with VSV-CoV-2 (MOI, 0.001 ffu/cell) for 1 h at 37° C. The mixture is added on cells for 1 h at 37° C. The monolayers are then washed and overlaid with medium containing 2% FBS for 18 h. The following day cells are fixed with paraformaldehyde 4%, stained with DAPI, and visualized using an ImageXpress Micro XL (Molecular Devices, San Jose, Calif., USA) microplate reader and a 10× S Fluor objective. The percentage of infected cells is estimated by counting the number of cells expressing GFP and the total number of cells (DAPI-positive cells) from four different fields per sample using MetaXpress software (Molecular Devices, San Jose, Calif., USA).

### D. Virucidal Assay

Viruses (10⁵ pfu of SARS-CoV-2) and test compounds are incubated for 1 h at room temperature, and then the virucidal effect is investigated by adding serial dilutions of the mixtures on Vero-E6 for 1 h, followed by addition of medium containing avicel. Viral titers are determined at dilutions at which the material is not effective.

### E. Results

The following compositions were tested as described above in Examples 4A, 3B and 3C:
MUSOT NP—a gold nanoparticle as described in Cagno et al., Nature Materials, DOI: 10.1038/NMAT5053 (2017);
MUS CD—a sulfonylalkylthio β cyclodextrin as described as “CD1” in Jones et al., Sci. Adv. 2020; 6: eaax9318 (2020);
6′SLN CD—shown as “C11-6′” in FIG. 4 of WO 2020/048976; and
SA11—a composition of the present invention, prepared, e.g., as described in Example 1.

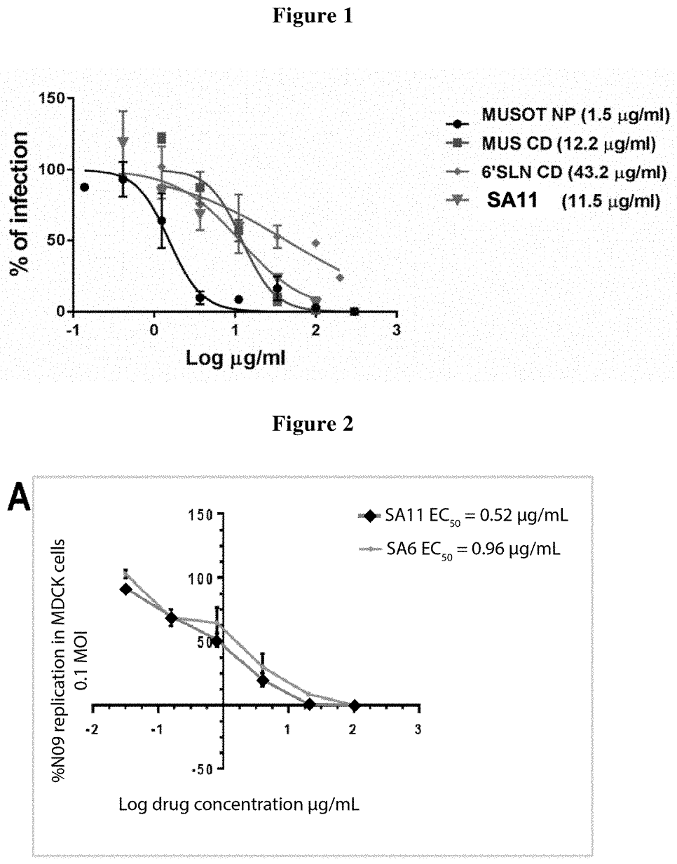

SA11 inhibited VSV-SarsCoV-2 Spike (a pseudo-virus containing the Spike S proteins of SARS-CoV-2) as shown in FIG. 1.

When tested as described above in Example 4D, the composition SA11 shows virucidal efficacy.

### Example 5

### Testing

### A. Materials

DMEM—Glutamax medium can be purchased from Thermo Fischer Scientific. Tween 20® for washing buffer and 3,3′-diaminobenzidine (DAB) tablets can be purchased from Sigma Aldrich. Primary antibody (Influenza A monoclonal antibody) can be purchased from Light Diagnostics. Secondary antibody (Anti-mouse IgG, HRP-linked antibody) can be purchased from Cell Signaling Technology®. The CellTiter 96® AQueous One Solution Cell Proliferation Assay that contains a tetrazolium compound [3-(4,5-dimethylthiazol-2-yl) (3-carboxymethoxyphenyl)-2-(4-sulfophenyl)-2Htetrazolium, inner salt; MTS] and an electron coupling reagent (phenazine ethosulfate; PES) can be purchased from Promega. Oseltamivir phosphate used in in vivo experiments can be obtained from Roche (Palo Alto, Calif.) as a powder and prepared in sterile water for oral gavage (PO) administration of 0.1 ml.

### B. Cell Culture

MDCK (Madin-Darby Canine Kidney Cells) cell line, can be purchased from ATCC (American Type Culture Collection, Rockville, Md.). The cells are cultured in Dulbecco's modified Eagle's medium with glucose supplement (DMEM+GlutaMAX™) containing 10% fetal bovine serum (FBS) and 1% penicillin/streptomycin. MDCK cell lines is grown in humidified atmosphere with CO2 (5%) at 37° C.

### C. Viral Strains

H1N1 Neth09 was a kind gift from Prof M. Schmolke (University of Geneva). All influenza strains are propagated and titrated by ICC on MDCK cells in presence of TPCK-treated trypsin (0.2 mg/ml)

### D. Inhibition Assays

MDCK cells are pre-plated 24 h in advance in 96-well plates. Increasing concentrations of materials are incubated with the influenza virus (MOI: 0.1) at 37° C. for one hour and then the mixtures are added to cells. Following the virus adsorption (1 h at 37° C.), the virus inoculum is removed, the cells are washed and the fresh medium is added. After 24 h of incubation at 37° C., the infection is analyzed with immunocytochemical (ICC) assay. The cells are fixed and permeabilized with methanol. Then the primary antibody (1:100 dilution) is added and incubated for 1 hour at 37° C. The cells are washed with wash buffer (DPBS+Tween 0.05%) three times; then secondary antibody (1:750 dilution) is added. After 1 hour the cells are washed and the DAB solution is added. Infected cells are counted and percentages of infection are calculated comparing the number of infected cells in treated and untreated conditions.

### E. Virucidal Assays

Viruses (focus forming unit (ffu):105/mL) and the materials (EC99 concentration) are incubated for 1 hour at 37° C. Serial dilutions of the virus-material complex together with the non treated control are conducted and transferred onto the cells. After 1 hour, the mixture is removed and the fresh medium is added. Next day, viral titers are evaluated.

### F. Results

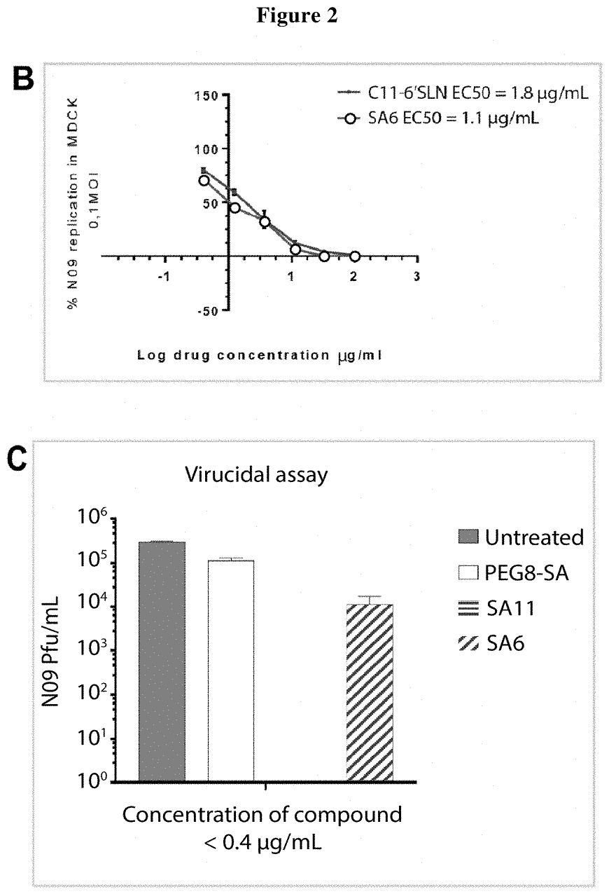

The following compositions SA11, SA6, PEG8 and C11-6′SLN (the compound C11-6′ as shown in FIG. 4 of WO 2020/048976) were tested as described above in Example 5. The results are shown in FIG. 2. FIG. 2A shows that both SA11 and SA6 inhibited H1N1 Neth09. FIG. 2B shows that SA6 inhibited H1N1 Neth09 with a lower EC₅₀ than the antiviral compound C11-6′SLN (the compound identified as C11-6′ in FIG. 4 of WO 2020/048976). FIG. 2C shows that SA11 and SA6 reduced the Pfu/mL by greater than an order of magnitude and are, therefore, virucidal; SA11 reduced the Pfu/mL so significantly that no bar can be seen in the corresponding column at the scale illustrated in FIG. 2C. While PEG8 did not give virucidal results in the test reported above, it cannot be concluded that PEG8 lacks antiviral activity useful in the invention.

### Example 6

### Resistance Testing

### A. Cells, Tissues, Viruses and Compounds

Calu-3 cells are cultured in MEM (Minimum Essential Medium) supplemented with Gluta MAX™, 10% FBS, Phenol Red, 1% Hepes, 1% Non-Essential Amino Acids, 1% penicillin/streptomycin and 1% Sodium-pyruvate and grow at 37° C. in an atmosphere of 5% CO2. MDCK cells are cultured in DMEM (Dulbecco's Modified Eagle Medium) supplemented with GlutaMAX™, Sodium Pyruvate, Phenol Red, 10% FBS and 1% P/S and grow at 37° C. in an atmosphere of 5% CO2. Human ex vivo reconstituted upper respiratory tissues, Mucilair, are purchased from Epithelix (Geneva, Switzerland) and handled according to the manufacturer's instructions.

Human H1N1, A/Netherlands/602/2009 Influenza virus (A(H1N1)PDM09), is amplified and titrated in MDCK cells by plaque assay. For viral stock production, the cells are infected with a multiplicity of infection (MOI) of 0.01 PFU/cell in serum-free DMEM, for 1 h at 37° C. The inoculum is then removed and fresh serum-free medium containing 1 μg/ml of TPCK trypsin is added. The infectious supernatant is collected 48 h hours post infection, aliquoted and frozen at −80° C. before titration. Viral stocks of the resistant variant against SA11, i.e., SA11p9 is prepared in Calu-3 cells with an MOI of 0.1 PFU/cell in serum-free MEM for 1 h at 37° C. The inoculum is removed, fresh serum-free medium is added and the infectious supernatant is collected at 48 h post infection, aliquoted and frozen at −80° C. before titration in MDCK cells.

### B. Cell Viability Assay

Calu-3 cells (1×1E5 cells per well) are seeded in a 96-well plate one day before the assay. A dose range of test composition (spanning from 125 ng/ml to 50 μg/ml), is added to the cell cultures in serum-free MEM for 24 or 48 hours. MTT reagent (Promega) is added to the cell cultures 3 h at 37° C. following to manufacturer instructions. Subsequently, the absorbance is read at 570 nm. Percentages of viability are calculated by comparing the absorbance in treated wells and untreated conditions.

### C. Infectivity of A(H1N1)pdm09 Influenza Viruses Measured by Plaque Assay in MDCK

The infectivity of the wild type (WT) virus and the selected variants is determined by at least two independent plaque assays. Confluent cultures of MDCK cells in 6 multi-well plates are incubated at 37° C. for 1 h with 10-fold serial dilutions of each virus strain prepared in serum-free minimal essential medium [MEM] containing 1% penicillin/streptomycin. Remove the inoculum from the cells, wash and add MEM containing 0.3% BSA, 0.9% Bacto agar, and 1 μg/ml TPCK-treated trypsin to the cell cultures. After 48 h of incubation at 37° C., the cells are fixed with 4% formaldehyde solution to be stained with 0.1% crystal violet. The number of PFU per dilution is determined using a finescale magnifying comparator and a white light table.

### D. Selection of Resistant Variants

(H1N1)pdm09 influenza viruses are successively passaged with an MOI of 0.1 PFU/cell in Calu-3 cells, seeded in 6 multi-well plates, in the presence of test composition and without test composition as a control for the effect of the cells on the variations found on the virus. Administer the first dose of test composition corresponding to its EC50 and double this value at each passage, until the toxic dose is reached (if there is a toxic dose). The cells are incubated with the compound for 48 h. Collect the supernatants, centrifuge at 3000 rpm for 5 minutes to separate dead cells from the viral suspensions. Infectious virus yields are determined as the number of PFU/ml in MDCK cells. The P values are calculated using the t test with a mathematical software such as Prism 8.0 (GraphPad, USA).

### E. Inhibition Assay

A dose range of test composition spanning from 1.2 μg/mL to 300 μg/mL is pre-incubated with 0.1 MOI of UTRp9 (9th passage without any compound) or N09 Stock for 1 hour in serum free DMEM at 37° C. Inoculate the virus stock and the compound (SA11) for 1 hour at 37° C. on a confluent layer of MDCK cells seeded in a 96 multi-well plate. The inoculum is then removed and the cells are overlaid with serum-free DMEM containing 1% penicillin/streptomycin. 12 hours post infection (hpi) at 37° C. the number of infected cells is calculated by immunocytochemistry. Fix the primary antibody in methanol (mouse monoclonal Influenza A antibody 1:100 dilution, Chemicon®) and keep it at 37° C. for 1 hour. The cells are then washed with DPBS/Tween 0.05% three times and the secondary antibody (Anti-mouse IgG, HRP-linked 1:750 dilution, Cell signalling technology) is added. After 1 hour the cells are washed and the DAB solution is added. Infected cells are counted and percentages of infection are calculated comparing the number of infected cells in treated and untreated conditions. Plot all results as the mean values from two independent experiments, performed in duplicate. The EC50 values for inhibition curves are calculated by regression analysis using a mathematical program such as GraphPad Prism version 8.0 (GraphPad Software, San Diego, Calif., U.S.A.).

### F. Results

When tested as described above in Example 5, influenza viruses do not develop resistance to SA11 and SA6.

1. (canceled)

2. (canceled)

3. (canceled)

4. (canceled)

5. A virucidal composition according to Formula (I) 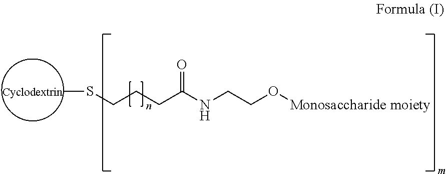
wherein
m is 2 to 8,
n is 2 to 28 or 4 to 13,
and sialic acid is the monosaccharide moiety.

6. The virucidal composition of claim 5, wherein cyclodextrin is selected from the group comprising alpha-cyclodextrin, beta-cyclodextrin, and gamma-cyclodextrin.

7. The virucidal composition of claim 5, wherein m is 3 or 4.

8. A virucidal composition according to Formula (II) 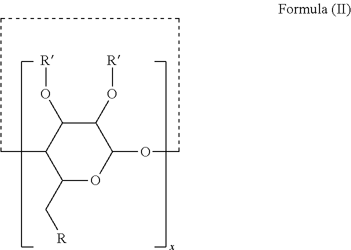
wherein:
each R, independently, is —OH, —SH or an optionally substituted alkyl-based ligand, wherein no more than 4 R groups can be —OH or —SH and at least two of said ligands comprise a sialic acid moiety;
each R′, independently, is H, —(CH₂)_{y}—COOH, —(CH₂)_{y}—SO⁻₃, a polymer or a water solubilizing moiety;
x is 6, 7 or 8; and
y is an integer from 4 to 20,
or a pharmaceutically acceptable salt thereof.

9. The virucidal composition of claim 8, wherein each R′ is H.

10. The virucidal composition of claim 8, wherein the alkyl-based ligands are optionally substituted C₆-C₁₅ alkyl-based ligands.

11. The virucidal composition of claim 8, wherein said optionally substituted alkyl-based ligands are selected from the group: alkylamidoalkoxy, alkylamidoalkylthio, carboxyalkyloxy, carboxyalkylthio.

12. A virucidal composition selected from SA11 and SA6, according to Formula (III): 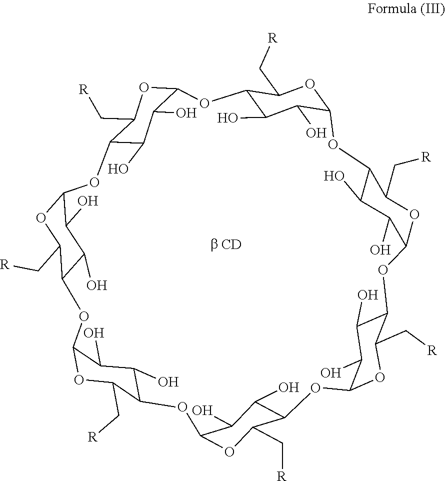
where each R is independently selected from —OH, —SH, Formula (IV), Formula (V), Formula (VI) and Formula (VII): 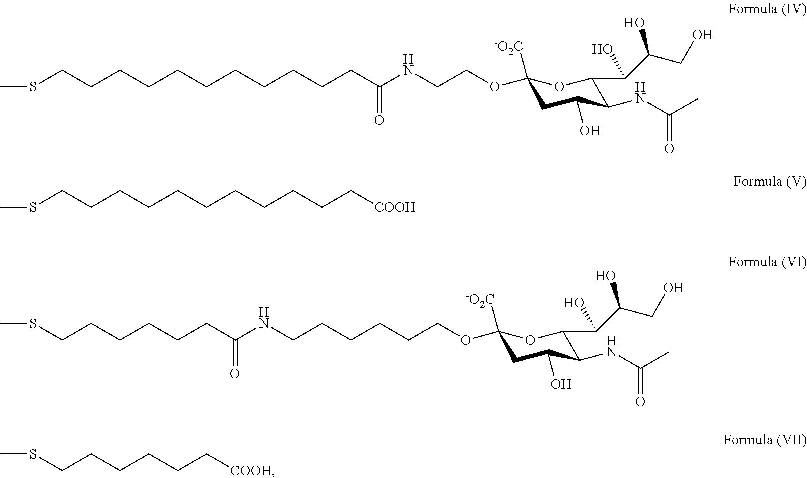
where, for SA11:
from 2 to 7 R groups are represented by Formula (IV), and
from 5 to 0 R groups are represented by Formula (V),
with 0, 1 or 2 R groups being —OH or —SH; and
for SA6:
from 2 to 7 R groups are represented by Formula (VI), and
from 5 to 0 R groups are represented by Formula (VII),
with 0, 1 or 2 R groups being —OH or —SH,
or a pharmaceutically acceptable salt thereof.

13. The virucidal composition of claim 12 that is SA11.

14. A pharmaceutical composition comprising an effective amount of one or more virucidal compositions of claim 5 and at least one pharmaceutically acceptable excipient, carrier and/or diluent.

15. The virucidal composition of claim 5 for use in treating a COVID-19 virus infection, an influenza infection, or a disease associated with a COVID-19 virus or influenza.

16. A virucidal composition comprising an effective amount of one or more virucidal compositions of claim 5 and optionally at least one suitable carrier or aerosol carrier.

17. A method of disinfection and/or sterilization comprising using the virucidal composition of claim 14.

18. A device comprising the virucidal composition of claim 14 and means for applying or dispensing the virucidal composition.

19. (canceled)
